(19) United States
(12) Patent Application Publication
(10) Pub. No.: US 2002/0099294 A1
(43) Pub. Date: Jul. 25, 2002

(54) Fluid cooled heat sensing device for thermal and skin burn evaluation

(76) Inventors: Hechmi Hamouda, Raleigh, NC (US); Roger L. Barker, Cary, NC (US); Darren W. Heath, Kannapolis, NC (US)

Correspondence Address: JENKINS & WILSON, PA, 3100 TOWER BLVD, SUITE 1400, DURHAM, NC, 27707, US

(21) Appl. No.: 09/938,236

(22) Filed: Aug. 23, 2001

Related U.S. Application Data
(60) Provisional application No. 60/228,684, filed on Aug. 29, 2000.

(51) Int. Cl. A61B 6/00

(52) U.S. Cl. 600/474

(57) ABSTRACT

A fluid cooled heat sensor device adapted to provide direct measurements of heat flux to be used for calculating thermal and skin burn predictions. The device comprises a copper disk which is inserted into an aperture in the top of a housing so as to define a fluid reservoir beneath the copper disk and within the housing. The housing further includes a fluid coolant entry port and fluid coolant outlet port for the ingress and egress of a suitable coolant fluid through the sensor device. At least one thermocouple is affixed to the back side of the copper disk and has an electrical connector wire extending therefrom and through the housing. The electrical connector wire is intended to be connected to suitable data collection equipment.

### DESCRIPTION

1. Technical Field

The present invention relates generally to heat sensor devices. More particularly, the present invention relates to an improved fluid cooled device for thermal and skin burn evaluation that utilizes direct measurement of heat flux in order to obtain precise heat flux measurement so as to determine accurate thermal and skin burn evaluation data.

2. Related Art

Laboratory test methods for evaluating the thermal protective performance (TPP) of clothing material must rely on instrumental measurements of the heat flux penetrating the test fabric and a mathematical model for translating thermal measurements to predict physiological skin burn injury. Over the past decade or more, several different types of sensor devices have been developed and used for this particular application. Although all of the previously developed devices have generally performed in accordance with at least minimal performance expectations, there has been a long-felt need for a new and improved sensing device for developing precise thermal and skin burn evaluations. Applicants have discovered such a heat sensor device and the device will be described in detail hereinafter.

First, as background, applicants wish to briefly describe the structure and functionality of four representative conventional sensors and one additional novel sensor used in the measurement of transient heat flux resulting from a flash-fire or steady heat source short exposure and assessment of resulting human skin burn damage in a laboratory test method environment. The specific application of the sensors is to evaluate the thermal protective performance (TPP) of clothing materials in the laboratory. In this respect, one previous sensor is well-known to those skilled in the art as the THERMOGAUGE™ sensor. The THERMOGAUGE™ sensor, available from Vatell Corporation of Blacksburg, Va., is a circular foil heat flux gauge that operates by measuring the temperature differential between the center and the circumference of a thin constantan foil disk. The constantan foil disk is bonded to a cylindrical copper heat sink, and the incident heat is drawn towards the heat sink away from the center of the constantan foil. This produces a temperature drop across the constantan foil which is measured by a thermoelectric junctions in the center of the constantan foil and the outer copper heat sink. The voltage output from the sensor is read and then combined with a calibration coefficient provided by the manufacturer to calculate the absorbed heat flux.

Another conventional sensor well-known to those skilled in the art is the HY-THERM® sensor available from Hy-Cal Sensing Products of El Monte, Calif. This sensor consists of an insulating wafer with a series of thermocouples embedded in the backside of the wafer in such a way that the thermoelectric junctions are positioned on opposite sides of the insulating wafer. The wafer is mounted to a heat sink that draws the incident heat. A temperature drop will result across the wafer and the thermocouples will respond to the temperature drop. The thermocouples are connected in series so as to provide an additive or amplified response in signal output. The signal output is then proportional to the heat flux incident upon the sensor.

Another conventional sensor is the TPP (Thermal Protective Performance) sensor, available from Custom Scientific Instruments, Inc., which comprises an insulated copper slug calorimeter. The TPP sensor is not cooled and has been proven in industrial applications as a rugged and reliable sensing device that is well established for use to measure heat flux measurements and predict human tissue damage.

Yet another conventional sensor well-known to those skilled in the art is the THERMOMAN™ sensor (also known as the “Embedded Thermocouple Sensor”). This type of sensor is currently in use (but soon to be replaced by the Pyrocal sensor described hereinafter) in a testing laboratory at the College of Textiles of North Carolina State University in Raleigh, N.C. on a full scale mannequin used to test flame retardant garments. The THERMOMAN™ sensor used in the mannequin testing of flame retardant garments is a thin-skin calorimeter which utilizes a T-type thermocouple which is buried below the exposed surface of a cast thermoset polymer resin plug at a depth of about 0.17 mm (0.005 inches). Scientists who work in the testing laboratory report that the polymer exhibits a thermal inertia similar to that of undamaged human skin. Thus, the Embedded Thermocouple Sensor is designed with a frontal thickness greater than 6.35 mm (0.25 inches) so that temperature conditions along the rear side of the sensor will not affect the response of the sensor surface measurements. This allows the sensor to be considered an infinite thickness slab utilizing the infinite slab geometry for the exposure. The depth of the thermocouple is critical to the analysis of heat flux in this sensor, and thus a computer program was used to calculate heat flux.

Finally, a fifth and novel sensor (Pyrocal) is described herein that has been developed at the College of Textiles of North Carolina State University and is the subject matter of co-pending and commonly assigned U.S. patent application Serial No. ______ filed ______ in the U.S. Patent and Trademark Office. The sensor is a copper disk within a copper thermal guard ring that are both supported within a heat insulating disk holder surrounded by a protective housing. A thermocouple is affixed to the back side of the copper disk in a cavity defined within the heat insulating disk holder, and a connector wire extends through the heat insulating disk holder and protective housing. This novel sensor is used in the testing described herein along with the four conventional sensors to evaluate the relative performance of the novel heat flux sensor of the present invention.

Most of the sensors described above possess certain disadvantages which has led to a long-felt need for an improved heat flux sensor device. Disadvantages of many previous heat flux sensors include known heat leakage from the sensor, limited durability, errors due to inaccurate thermocouple bead location, polymer cracks with repetitive testing exposures and undesirably large and bulky housings required to insulate sensors against heat loss. These shortcomings and others have been overcome by the novel fluid cooled heat flux sensor discovered by the applicants and described and claimed herein.

### SUMMARY OF THE INVENTION

In accordance with the present invention applicants have discovered a novel fluid cooled heat sensing device adapted for direct measurement of heat flux and comprising a copper disk having a front side and a back side, and a housing having an open top and a closed bottom with a fluid reservoir therein. The open top of the housing is adapted to sealingly receive the copper disk therein with the front side of the copper disk facing outwardly and the fluid reservoir disposed adjacent the back side of the copper disk and within the housing. A fluid coolant entry aperture and a fluid coolant outlet aperture are provided in the housing, and one or more thermocouples are affixed to the back side of the copper disk with electrical connector wires extending from the thermocouples and through the housing.

Therefore, it is an object of the present invention to provide a fluid cooled heat sensor device for accurately measuring transient heat flux resulting from flash fire or a steady heat source short exposure so as to reliably assess resulting skin burn damage potential.

It is another object of the present invention to provide a fluid cooled heat sensor device that allows for direct measurement of heat flux as opposed to an indirect measurement of heat flux in order to provide a more accurate assessment of potential skin burn damage during garment flammability testing.

It is still another object of the present invention to provide a fluid cooled heat sensor device that provides a stable output over a wide range of thermal exposures of interest in laboratory testing of garment flammability and that is rugged and economical in use.

It is still another object of the present invention to provide a fluid cooled heat sensor device that is highly sensitive to heat flux variations and that does not require any calibration and that does not suffer from drifts in response output.

It is still another object of the present invention to provide a fluid cooled heat sensor device that can be used in a substantially unlimited heat range for accurately measuring transient heat flux and that is relatively small in size in view of the high sensitivity capability thereof for measuring transient heat flux.

Some of the objects of the invention having been stated, other objects and advantages of the inventive fluid cooled heat sensor device will become apparent as the description proceeds and when taken in connection with the accompanying drawings as described hereinbelow.

### BRIEF DESCRIPTION OF THE DRAWINGS

FIG. 1 is a perspective view of the fluid cooled heat sensor device of the present invention;

FIG. 2 is a vertical cross-sectional view of the fluid cooled heat flux sensor device shown in FIG. 1;

FIG. 3 is a top plan view of the copper disk and the housing of the fluid cooled heat sensor device of the present invention;

FIG. 4 is a schematic view of the fluid cooled heat sensor device in a system including a cooling auxiliary for providing cooled fluid to the sensor device and a data collection apparatus for collecting heat flux measurement data from the sensor device for use in predicting human skin burn damage potential;

FIG. 5 is a view of a RPP (Radiant Protective Performance) test stand;

FIG. 6 is a graph of the performance of the fluid cooled heat flux sensor device shown in FIG. 1 and five other sensors when exposed to 2.5 kW/m² heat flux level for 5 minutes;

FIG. 7 is a graph of the performance of the fluid cooled heat flux sensor device shown in FIG. 1 and three other sensors when exposed to 6.3 kW/m² heat flux level for 5 minutes;

FIG. 8 is a graph of the performance of the fluid cooled heat flux sensor device shown in FIG. 1 and three other sensors when exposed to 9.6 kW/m² heat flux level for 5 minutes; and

FIG. 9 is a table of the performance of the fluid cooled heat flux sensor device shown in FIG. 1 and four other sensors regarding predicted time to second degree burn based on performance data gathered at 6.3 kW/m² and 9.6 kW/m² heat flux levels.

### BEST MODE FOR CARRYING OUT THE INVENTION

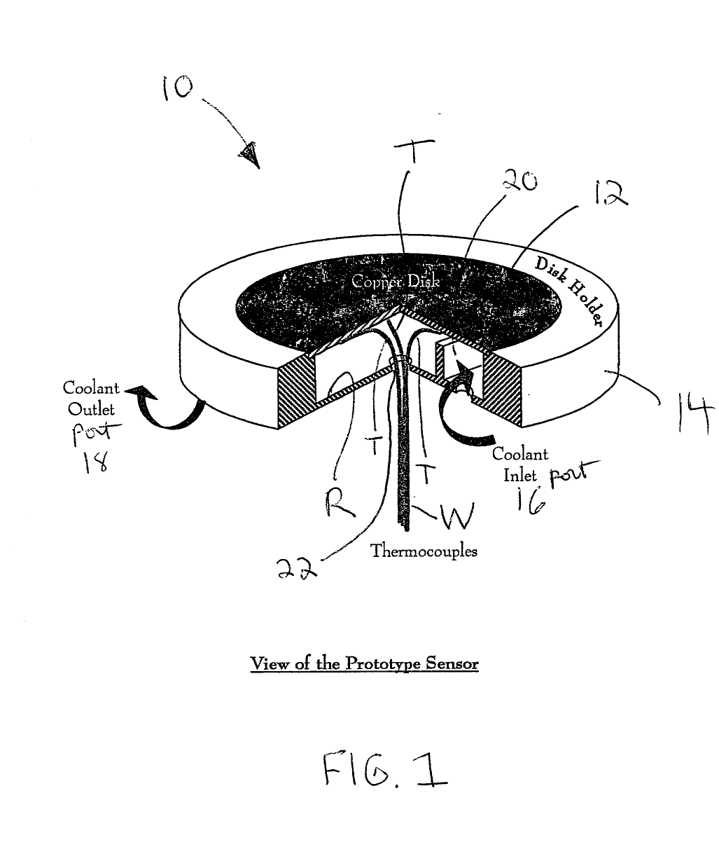

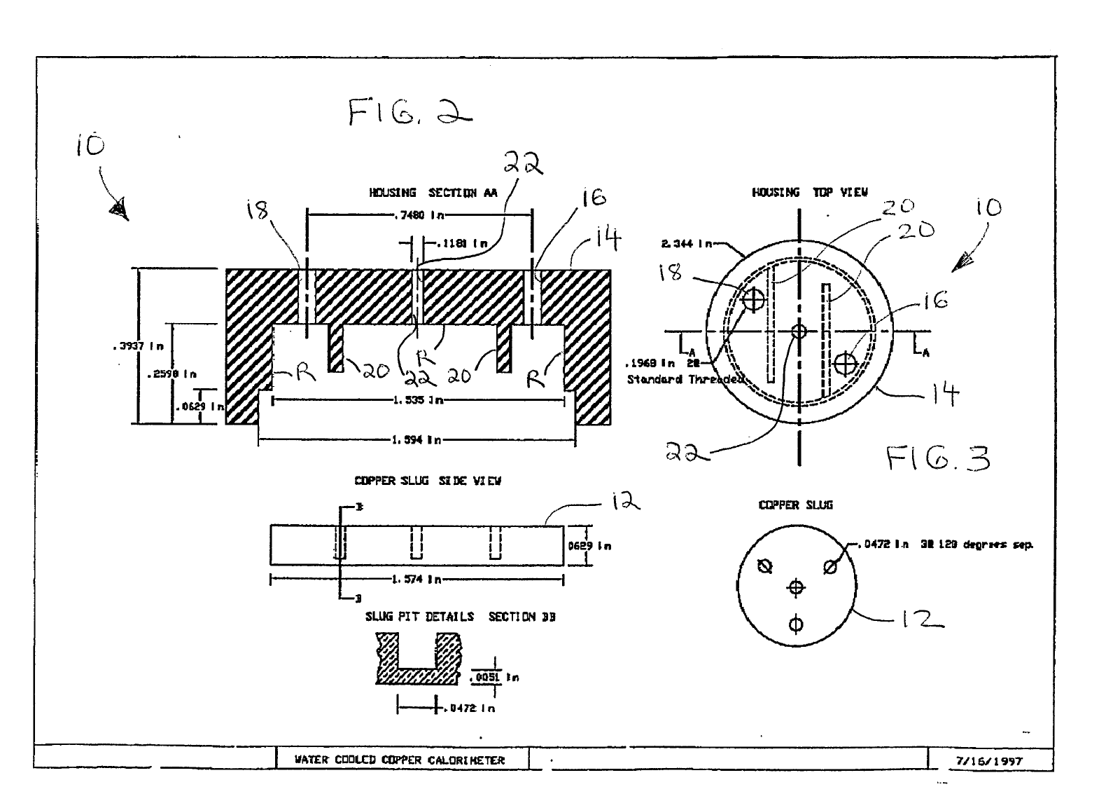

Referring now to FIGS. 1-9 of the drawings, the fluid cooled heat flux sensor of the present invention is shown and designated 10. Fluid cooled heat sensor 10 is a liquid cooled heat flux sensor that is particularly well adapted to evaluate fire protective clothing in extended time and low heat flux exposure testing. The liquid coolant can be tap water and is preferably distilled water.

As is well known to those skilled in the art, heat flux is a measurement of the amount of thermal energy passing through an area (w/m²) and extended time describes the duration of time in which fluid cooled sensor 10 will be operated during an exposure (e.g., 300 seconds in testing described hereinafter). Low heat flux is considered values ranging between 2.5 kW/m² and 9.6 kW/m². Direct exposure of fluid cooled heat sensor 10 as described hereinafter will consist of exposure of an individual fluid cooled heat sensor 10 directly to a heat source, and RPP (Radiant Protective Performance) exposure will consist of exposure of an individual fluid cooled heat sensor 10 to a heat source through a test fabric. As will be appreciated from the detailed description set forth below, fluid cooled heat sensor 10 is particularly well adapted to be used for extended time heat flux exposures and is responsive to conductive, convective and radiant heat transfer which will produce a steady, reliable output over extended time in low heat flux conditions.

Fluid cooled heat sensor 10 is a slug-type thermal sensor developed by applicants for use in flame retardant garment testing on a test mannequin at the College of Textiles of North Carolina State University in Raleigh, N.C. Sensor 10 comprises a thin copper disk 12, preferably between about 1.570 and 1.590 inches in diameter and 0.625 and 0.630 inches in thickness, and most preferably about 1.574 inches in diameter and 0.0629 inches in thickness. Copper disk 12 is configured so as to be inserted sealingly into housing 14 (which is preferably also formed of copper). Housing 14 has a closed bottom and an open top into which copper disk 12 is inserted. Housing 14 further defines a fluid reservoir R therein having a fluid coolant entry port 16 and a fluid coolant outlet port 18 in the bottom surface. Fluid cooled inlet port 16 allows for the introduction of a cooled fluid into heat sensor 10 and fluid coolant outlet port 18 allows for the cooled fluid to be removed from sensor 10 so that a continuous cooled fluid flow may be maintained from a cooling auxiliary such as a small pump (see FIG. 4). Thus, it can be appreciated and understood by one skilled in the art that fluid cooled sensor device 10 will be in fluid connection in a closed fluid coolant loop with a suitable cooling auxiliary.

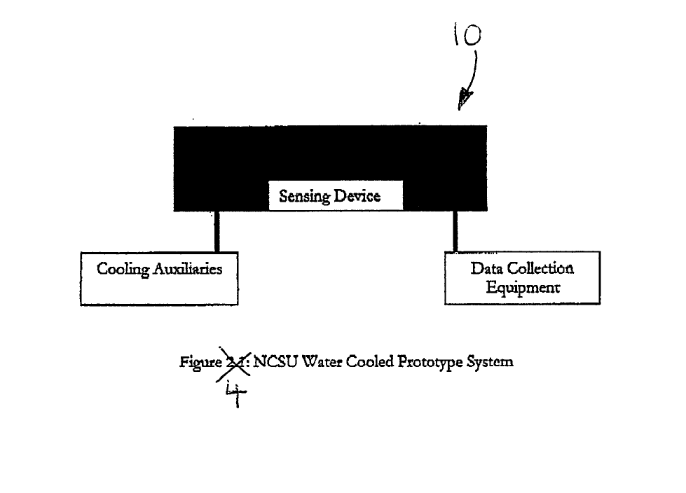

Also, housing 14 is provided with two upstanding divider walls 20 therein which extend upwardly from the bottom of housing 14 but terminate prior to contact with copper disk 12. As can be seen with reference to FIG. 3, two divider walls 20 are provided adjacent coolant inlet port 16 and coolant outlet port 18, respectively, in order to enlarge upon the flow path of fluid coolant through sensor 10 in order to facilitate the distribution of the fluid coolant throughout reservoir R beneath copper disk 12. The enhanced distribution of the fluid coolant provides for greater accuracy of sensor 10.

Also, at least one, and preferably three, thermocouples T are attached to the back side of copper disk 12 within reservoir R. Thermocouples T are most suitably a T-type thermocouple, and have a connector wire W attached to each thermocouple T. Connector wires W extend downwardly from copper disk 12 and through reservoir R and finally through an aperture 22 in the bottom of housing 14 of sensor 10. Connector wires W from thermocouples T are in electrical connection with data collection equipment such as a data acquisition system (see FIG. 4) which serve to collect the heat flux measurements for calculating potential human skin burn damage.

### Water Cooled Prototype Sensor

The thermal sensing system shown in FIG. 4 and incorporating sensor 10 comprises water-cooled sensor 10, and cooling auxiliaries and data collection equipment. The sensor assesses incident heat flux by measuring the temperature of water flowing through the system. The temperature rise in the coolant is calibrated to known levels of incident heat flux.

A mathematical method was developed to calculate heat flux using temperatures, heat transfer coefficients and the physical properties of the system.

### Mathematical Model For Calculating Heat Flux

The following temperature information was needed in order to calculate heat flux: temperature in the copper disk; water temperature coming in and out of the system; and temperature of the housing which contains the copper disk and the coolant. The following formula describes the heat flux calculation used for the prototype sensor:

(Total Heat Flux)=(Energy Stored in Copper Disk)+(Energy Stored in Water)−(Energy Stored in Housing)

or symbolically,

$$q = {ρ}_{CU} {C}_{Pcu} {t}_{CU} {A}_{CU} \frac{ⅆ {T}_{CU}}{ⅆ t} + {\dot{m}}_{H20} {C}_{{P}_{H20}} ( {T}_{I} - {T}_{O} ) - {h}_{H} {A}_{H} ( {T}_{H} - {T}_{I} ) \qquad [ A ]$$

where,

ρ_{CU} is the density of the copper disk (kg/m³)

C_{pCU} is the specific heat of the copper slug (kJ/kg*K)

t_{CU} is the thickness of the copper slug (m)

A_{CU} is the area of the copper disk (m²)

T_{CU} is the temperature of the copper slug (K°)

t is the time step of the experiment (sec)

\dot{m}_{H20} is the mass flow rate of the coolant (kg/sec)

C_{pH20} is the specific heat of the coolant (kJ/kg*K)

h_{H} is the heat transfer coefficient of the sensor housing (kJ/sec*m²*K)

A_{H} is the wetted area of the sensor housing (m²)

Three major phenomenon occur within the prototype sensor designated by the three terms on the right hand side of equation [A]. The first is the transfer of energy through the copper disk represented in the following equation:

$${ρ}_{CU} * {C}_{PCU} * {t}_{CU} * {A}_{D} * \frac{ⅆ {T}_{CU}}{ⅆ t} = {q}_{CU} \qquad [ B ]$$

where q_{CU} equals the energy stored by the copper disk.

The second component represents the absorption of energy by the coolant as determined by the following equation:

\dot{m}_{H20}*C_{PH20}*dT_{H20}=q_{H20} [C]

Where q_{H20} represents the energy stored in the coolant.

The third component represents the transfer of energy from the sensor housing into the coolant represented by the following equation:

h_{H}*A_{H}*(T_{H}−T_{I})=L [D]

Where L represents the energy stored in the housing or losses of the system. The transfer of energy from the housing into the water is labeled “losses” and can be accounted for by calculating heat transfer coefficients, equation [E] and equation [F], which governs the rate at which the energy is transferred into the coolant.

$$\frac{{A}_{CU}}{{A}_{H}} * \frac{1}{( {T}_{H} - {T}_{A} )} [ ( \frac{{ρ}_{CU} * {C}_{PCU} * {t}_{CU} * ⅆ {T}_{CU}}{ⅆ t} ) + ( {h}_{CU} ( {T}_{CU} - {T}_{A} ) ) - q ] = {h}_{H} \qquad [ E ]$$

Where h_{H} represents the heat transfer coefficient of the housing.

$${q}_{H20} / ( {T}_{D} - {T}_{A} ) = {h}_{CU} \qquad [ F ]$$

Where h_{CU} represents the heat transfer coefficient of the copper disk.

By running some preliminary tests and manipulating the above equations, all three phenomenon discussed above (Equation B, C and D) can be derived into one equation below. This equation was used extensively throughout the remainder of the test. Table 1 shows the numerical results of the heat transfer coefficients for 3 different exposure conditions of 2.5, 6.3, and 9.66 kW/m² using a constant coolant mass flow rate. Values of the thermal properties and geometrical parameters used in calculating the heat flux using equation [G] are shown in Table 2.

$$q = {ρ}_{CU} {C}_{PCU} {t}_{CU} \frac{ⅆ {T}_{CU}}{ⅆ t} + {h}_{CU} ( {T}_{D} - {T}_{I} ) - {h}_{H} \frac{{A}_{H}}{{A}_{CU}} ( {T}_{H} - {T}_{I} )$$

TABLE 1
Heat transfer coefficients for the copper disk h_{cu} and of the sensor
housing h_{H}.

| | Heat Transfer | Heat Transfer |
|---|---|---|
| | Coefficient of Copper | Coefficient of Sensor |
| kW/m² | Disk h_{cu} | Housing h_{H} |
| 2.5 | 3111 | 1406 |
| 6.3 | 2871 | 1098 |
| 9.6 | 3259 | 1160 |
| Average | 3080 | 1221 |

TABLE 2
Numerical constants used in calculating the heat flux using equation
[G] of the liquid cooled prototype sensor.

| Q_{CU} | C_{PCU} | t_{CU} | A_{CU} | h_{CU} | A_{H} | h_{H} |
|---|---|---|---|---|---|---|
| (kg/m³) | (j/kg*K) | (m) | (m²) | (W/m²*K) | (m²) | (W/m²*K) |
| 8954 | 381 | .001524 | 0.00119 | 3080.62 | 0.0015 | 1221.11 |

### Experimental Testing

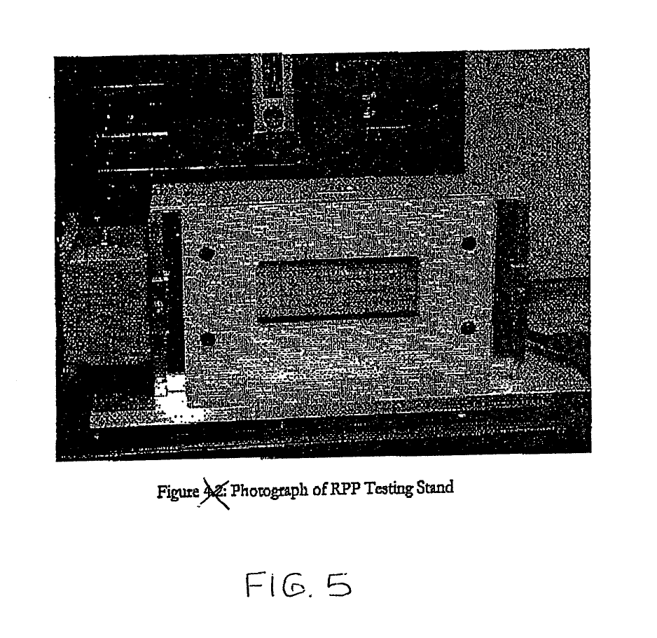

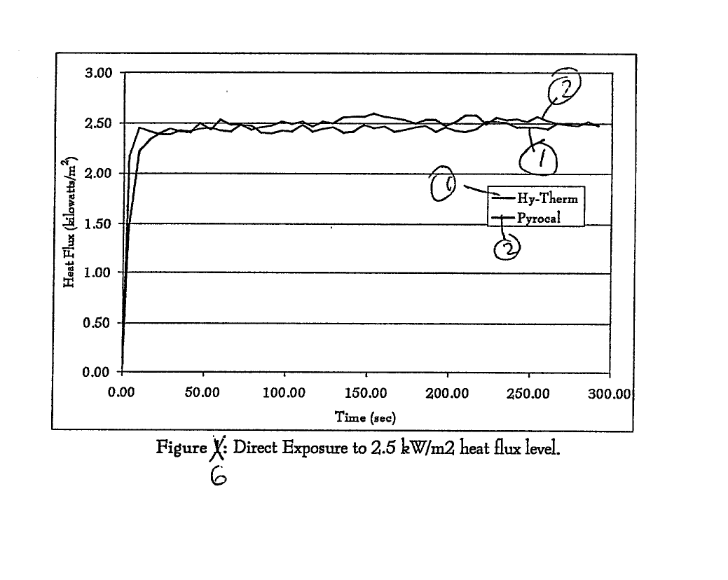

In conducting a comparative study of the performance of different sensors, an RPP (Radiant Protective Performance) test platform was used. A view of the RPP testing stand can be seen in FIG. 5. The RPP contains a mounting assembly that is 5.0 inches by 5.0 inches by 2.0 inches high. It uses quartz radiant heater tubes to provide a stable heat source. The RPP tester utilizes a heat shield that acts as a barrier prior to starting a test exposure.

### Direct Exposure

To compare their performance and response accuracy, the six sensors (including fluid cooled sensor 10) were directly exposed, for 5 minutes, to a 2.5 kW/m²heat flux level that approximates the range commonly sensed behind thermal protective fabrics. The evaluated sensors, as previously described, were the THERMOGAUGE™; HY-THERM®; Pyrocal novel sensor; TPP; THERMOMAN™; and fluid cooled heat sensor 10 of the invention.

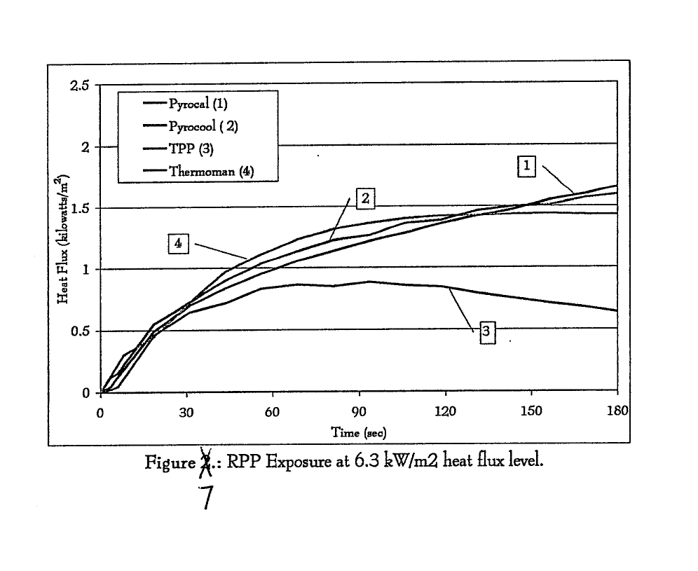

During the exposure, as shown in FIG. 6, both sensor 10 and TPP sensors have the shortest response time. However, as the exposure time elapses and within 20 seconds the temperature response of these two sensors drifts apart and away from the responses of the remaining sensors. Besides the THERMOGAUGE™ sensor, the remaining three sensors accurately track the incident heat flux level up to 2 minutes of exposure. At this time, the THERMOMAN™ sensor response starts drifting down apart from the response of the remaining sensors. Toward the end of the 5 minutes exposure time, both HY-THERM™ and the water cooled heat sensor 10 are still accurately tracking the incident heat flux. In spite of its steady constant response, the THERMOGAUGE™ sensor consistently generates a low reading of the incident heat flux.

This exposure based on a known heat flux level sets the needed performance confirmation of the different sensors. It shows that, up to approximately 2 minutes of exposure, three sensors: HY-THERM®, water cooled sensor 10 and THERMOMAN™ perform comparatively in tracking the incident heat flux level. Beyond the 2 minutes period only the HY-THERM® and water cooled sensor 10 remain generating a steady response throughout the 5 minutes of exposure.

### RPP (Radiant Protective Performance) Exposure

Four out of the previous six sensors were used in an RPP exposure test setup (see FIG. 5) with a composite fire fighter fabric system inserted between the heat source and the sensors. The HY-THERM® sensor was eliminated from this experiment to prevent damage due to fabric degradation residues. The THERMOGAUGE™ sensor was also eliminated for its consistent low reading of the heat flux level. Two heat flux levels of 6.3 and 9.6 kW/m² were used during this experiment that was conducted to evaluate the sensors' response to heat flux through fabric systems and predict the time to second degree burn based on each individual sensor response.

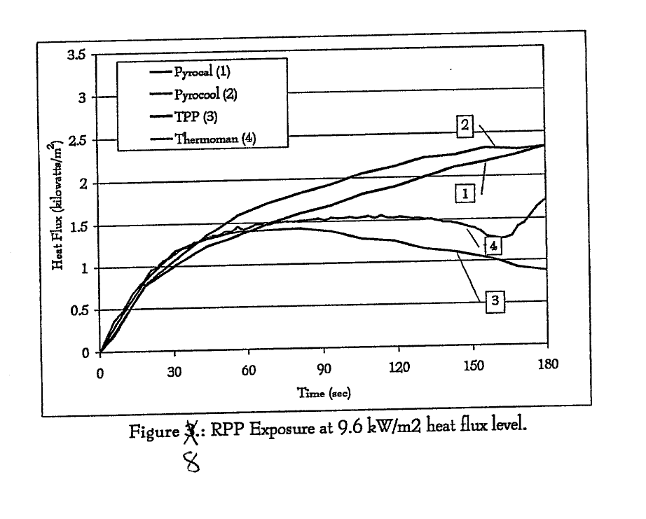

At the 6.3 kW/m² level, as shown in FIG. 7, apart from the THERMOMAN™ sensor which generates a higher response throughout the first 2 minutes of exposure, water cooled sensor 10 exhibits the shortest response time followed by the Pyrocal sensor and then the TPP sensor. However, as the exposure time elapses and within 1 minute both responses of sensor 10 and TPP sensors start drifting apart whereas water cooled sensor 10 continues tracking the sensed heat flux. Beyond the first 2 minutes of exposure, the THERMOMAN™ sensor starts its downward trend due to sensor heat storage.

When exposed to the next heat flux level of 9.6 kW/m², as shown in FIG. 8, the response time and the heat flux readings of all four sensors are comparable during the initial 30 seconds of exposure except for the THERMOMAN™ sensor that generates a higher response. For the remaining exposure time, both the Pyrocal sensor and the TPP sensors drift apart and away from the responses fo the remaining sensors. The trends of both water cooled sensor 10 and the Pyrocal sensor are similar to those exhibited during the previous exposure.

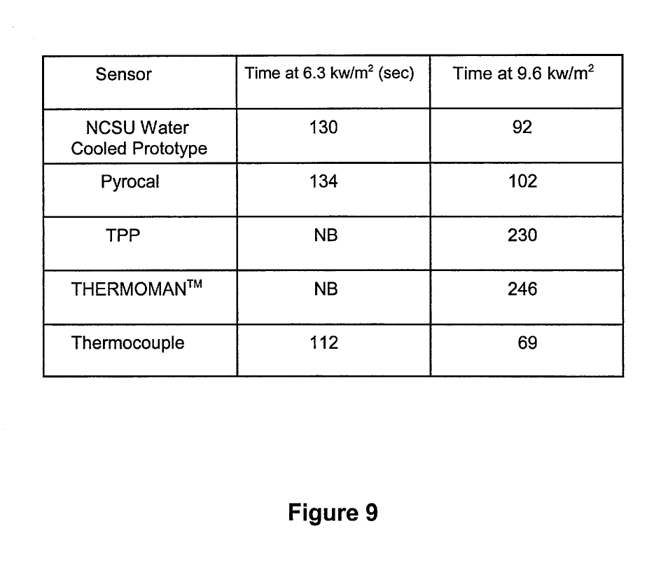

From these results and an additional temperature measurement based on a conventional thermocouple attached to the backside of the fabric, time to second-degree burn was calculated based on the Stoll's criteria for the Pyrocal sensor; water cooled sensor 10; and TPP sensors. A burn prediction program was used to determine the time to second-degree burn forthe THERMOMAN™ sensors. The 55° C. criterion was used in association with the thermocouple data. FIG. 9 shows these results as predicted with the five different sensors (including the thermocouple additional temperature measurement). At the 6.3 kW/m² heat flux level, the TPP sensor predicts no second-degree burn. Meanwhile, the THERMOMAN™ sensor predicts the longest time to second degree burn, 284 seconds, followed by the Pyrocal sensor, water cooled sensor 10 and finally the thermocouple which predict the shortest time of 112 seconds. The trend is the same at the 9.6 kW/m² heat flux level, the TPP sensor predicts the longest time to second degree burn, 230 seconds, while 69 seconds is the shortest time as predicted by the thermocouple. Results obtained based on the readings of both sensor the Pyrocal sensor and water cooled sensor 10 are in agreement.

Summarily, since it was shown and verified that both the Pyrocal sensor and the water cooled sensor 10 closely track the incident heat flux during for at least the initial 2 minutes of direct exposure, the final prediction of the time to second-degree burn based on these two sensors should be the most accurate. Additionally, both these predictions were obtained based on a direct reading of heat flux from the fabric surface opposite to the heat source while other sensors including the THERMOMAN™ and the thermocouple rely on indirect methods of heat flux evaluation or burn time prediction.

Applicants wish to note that although a specific application of water cooled sensor 10 is described herein, the applicants contemplate many other applications for water cooled sensor 10 and intend for all applications to be within the scope of the invention. Further, applicants again note that the newly-discovered Pyrocal sensor described above is also not a conventional heat flux sensor although it is included in the tests described herein as an additional data source. It is, in fact, novel and the subject matter of co-pending and commonly assigned U.S. patent application Ser. No. ______ filed ______.

It will be understood that various details of the invention may be changed without departing from the scope of the invention. Furthermore, the foregoing description is for the purpose of illustration only, and not for the purpose of limitation—the invention being defined by the claims.

What is claimed is:

1. A heat sensor device adapted for direct measurement of heat flux and comprising:
(a) A copper disk having a front side and a back side;
(b) a housing having an open top and a closed bottom and having a fluid reservoir therein, said open top being adapted to sealingly receive said copper disk therein with the front side of said copper disk facing outward and said fluid reservoir disposed adjacent the back side of said copper disk and within said housing;
(c) a fluid coolant entry aperture and a fluid coolant outlet aperture in said housing; and
(d) at least one thermocouple affixed to the back side of said copper disk and having an electrical connector wire extending from said thermocouple and through said housing.

2. The heat sensor device according to claim 1 wherein said copper disk is about 1.574 inches in diameter and 0.0629 inches in thickness.

3. The heat sensor device according to claim 1 wherein said housing is formed of a heat resistant material such as copper.

4. The heat sensor device according to claim 3 wherein said housing includes a plurality of walls therewithin for impeding coolant flow through said reservoir.

5. The heat sensor disk device according to claim 1 wherein said thermocouple is a T-type thermocouple.

6. The heat sensor disk device according to claim 5 wherein said at least one thermocouple comprises three thermocouples.

7. The heat sensor disk device according to claim 1 wherein said thermocouple electrical connector wire extends from said thermocouple affixed to the back side of said copper disk and through said fluid reservoir and said housing and outwardly from said protective housing.

8. The heat sensor disk device according to claim 1 including a cooling apparatus in fluid connection with said fluid coolant entry and outlet apertures of said heat sensing device.

9. The heat sensor device according to claim 8 including data collection apparatus in electrical connection with said plurality of thermocouples of said heat sensing device.

10. A heat sensor device adapted for direct measurement of heat flux and comprising:
(a) A copper disk having a front side and a back side;
(b) a housing having an open top and a closed bottom and having a fluid reservoir therein, said top being adapted to sealingly receive said copper disk therein with the front side of said copper disk facing outward and said fluid reservoir disposed adjacent the back side of said copper disk and within said housing;
(c) a fluid coolant entry aperture and a fluid coolant outlet aperture in said housing;
(d) a plurality of thermocouples affixed to the back side of said copper disk and having corresponding electrical connector wires extending from said thermocouples and through said housing; and
(e) a plurality of walls within said housing for impeding coolant flow through said fluid reservoir of said housing.

11. The heat sensor device according to claim 10 wherein said copper disk is about 1.574 inches in diameter and 0.0629 inches in thickness.

12. The heat sensor device according to claim 10 wherein said housing is formed of a heat resistant material such as copper.

13. The heat sensor disk device according to claim 10 wherein said thermocouples are T-type thermocouples.

14. The heat sensor device according to claim 10 including a cooling apparatus in fluid connection with said fluid coolant entry and outlet apertures of said heat sensor device.

15. The heat sensor device according to claim 14 including data collection apparatus in electrical connection with said plurality of thermocouples of said heat sensor device.
